(19) United States
(12) Patent Application Publication
(10) Pub. No.: US 2021/0008142 A1
(43) Pub. Date: Jan. 14, 2021

(54) PREPARATION AND USES OF SPINOSIN-Na FROM ZIZIPHI SPINOSAE SEMEN

(71) Applicant: Shanxi University, Taiyuan (CN)

(72) Inventors: Zhuoyu LI, Taiyuan (CN); Shuhua SHAN, Taiyuan (CN); Guisheng SONG, Taiyuan (CN); Yue XIE, Taiyuan (CN); Hanqing LI, Taiyuan (CN); Bin JIA, Taiyuan (CN); Jiangying SHI, Taiyuan (CN)

(21) Appl. No.: 16/740,474

(22) Filed: Jan. 12, 2020

(30) Foreign Application Priority Data
Jul. 9, 2019 (CN) 201910613520.2

(51) Int. Cl. A61K 36/72; A61P 35/00

(52) U.S. Cl. CPC A61K 36/72; A61P 35/00; A61K 2236/333; A61K 2236/39; A61K 2236/15

(57) ABSTRACT

Disclosed are a preparation method and applications of a spinosin-Na monomer of Ziziphi Spinosae Semen. The preparation method includes: pulverizing Ziziphi Spinosae Semen; hydrolyzing the powder with an acidic hydrolysis solution; neutralizing the hydrolysate; extracting the supernatant with ethyl acetate several times to produce a crude extract; and separating the crude extract sequentially using macroporous resin and HPLC to produce the spinosin-Na monomer of Ziziphi Spinosae Semen with a good water solubility and a purity greater than 98%. The spinosin-Na monomer prepared herein is capable of significantly inhibiting the proliferation of tumor cells, so that it can be applied in the preparation of a functional food and/or medicine for preventing and/or treating tumors.

### CROSS-REFERENCE TO RELATED APPLICATIONS

This application claims the benefit of priority from Chinese Patent Application No. 201910613520.2, filed on Jul. 9, 2019. The content of the aforementioned application, including any intervening amendments thereto, is incorporated herein by reference in its entirety.

### TECHNICAL FIELD

The application relates to the preparation of medicinal active ingredients of Ziziphi Spinosae Semen, and more particularly to spinosin-Na, and a preparation method thereof and applications thereof in the preparation of functional foods and/or medicines for preventing and/or treating tumors.

### BACKGROUND OF THE INVENTION

Ziziphi Spinosae Semen, dried and mature seed of Ziziphus jujube Mill. var. spinosa pertaining to Rhamnaceae, is edible and medicinal. Ziziphi Spinosae Semen plays a role in tonifying the liver, calming the heart, arresting sweating and promoting the production of body fluid, so in ancient China, Ziziphi Spinosae Semen was generally served as a good traditional Chinese medicine for sedation and hypnosis, and refreshing and tranquilizing. Ziziphi Spinosae Semen is mainly produced in Shanxi, Hebei, Shaanxi, Liaoning and Henan provinces in China, and contains a wide variety of active ingredients, such as fatty acids, triterpenoids, polyphenols, alkaloids and amino acids, which facilitate the sedation and hypnosis. It has been found that the main components of polyphenols from Ziziphi Spinosae Semen are spinosin and its derivatives, which can exert sedative, hypnotic and antidepressant effects.

Currently, several methods have already been reported to prepare spinosin from Ziziphi Spinosae Semen, but the spinosin prepared by the existing methods has poor water-solubility, leading to low bioavailability. By optimizing the existing preparation processes, this invention prepares spinosin-Na with good water solubility and a purity of greater than 98%. Moreover, the spinosin-Na is discovered for the first time to have significant antitumor effects without damaging the normal cells and the antitumor activity is significantly higher than spinosin. Therefore, the invention provides a preparation method of spinosin-Na and an application of spinosin-Na in the preparation of functional foods and/or medicines for preventing and/or treating tumors.

### SUMMARY OF THE INVENTION

This invention aims to provide a method of preparing a spinosin-Na monomer from Ziziphi Spinosae Semen to overcome the defects in the prior art, which has simple and time-saving process, and is suitable for large-scale production. The spinosin-Na monomer obtained herein has good water solubility and high purity, and can be used as a reference standard. Moreover, the spinosin-Na also has remarkable anti-tumor effect, so that it can be applied in the preparation of functional foods and/or medicines for preventing and/or treating tumor diseases.

The technical solutions of this invention are described as follows.

In a first aspect, the invention provides a spinosin-Na monomer of formula (I):

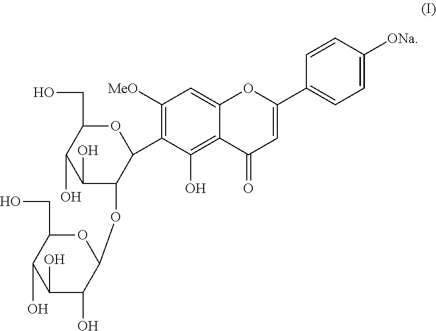

In a second aspect, the invention provides a method of preparing a spinosin-Na monomer from Ziziphi Spinosae Semen, comprising:

(1) Preparing a crude polyphenol extract of Ziziphi Spinosae Semen according to Chinese Patent Application No. 201810038716.9, wherein the preparation of the crude polyphenol extract of Ziziphi Spinosae Semen specifically comprises:

Pulverizing Ziziphi Spinosae Semen; hydrolyzing the pulverized Ziziphi Spinosae Semen with an acidic hydrolysis solution; neutralizing the hydrolysate with a NaOH solution to terminate the hydrolyzation; centrifuging the neutralized product and discarding the precipitate; extracting the supernatant with ethyl acetate followed by centrifugation to collect an aqueous phase as the crude polyphenol extract of Ziziphi Spinosae Semen; wherein a polyphenol content in the crude polyphenol extract of Ziziphi Spinosae Semen is measured by Folin-Ciocalteu assay;

(2) Statically adsorbing the crude polyphenol extract of Ziziphi Spinosae Semen with a macroporous resin; eluting the macroporous resin with ultrapure water and 30% acetone three times, respectively; eluting the macroporous resin with 50% acetone to collect an eluate; concentrating the eluate by rotary evaporation at 60° C. until the eluate is decreased by 75% in volume; and drying the concentrated eluate under vacuum to produce polyphenol powder of Ziziphi Spinosae Semen (ZSSP);

Wherein the macroporous resin is an SP207 macroporous resin, and a static adsorption time is 12-16 h;

(3) Dissolving the ZSSP powder obtained in step (2) in 40% methanol to produce a ZSSP solution; finely separating the ZSSP solution using HPLC to collect a fraction with a retention time of 22.444 min; concentrating the fraction by rotary evaporation at 60° C., and lyophilizing the concentrated fraction to obtain the spinosin-Na monomer of Ziziphi Spinosae Semen.

The HPLC separation is performed using a Phenomenex Hydro-RP C18 column (250 mm×10 mm, 4 μm) with a mobile phase of 40% methanol, a flow rate of 1 mL/min and a detection wavelength of 330 nm.

An appropriate amount of spinosin-Na is dissolved in ultrapure water, and then measured by MTT assay for the antitumor activity. The results show that the spinosin-Na can significantly inhibit the proliferation of various tumor cells and induce the apoptosis of tumor cells.

In a third aspect, the invention provides a use of the spinosin-Na in the preparation of a medicine for preventing and/or treating tumors.

In a fourth aspect, the invention provides a use of the spinosin-Na in the preparation of a functional food for preventing and/or treating tumors.

In a fifth aspect, the invention provides a use of the spinosin-Na as a reference standard.

Compared to the prior art, the invention has the following beneficial effects.

The method of the invention has a simple extraction process and the prepared spinosin-Na monomer is easily soluble in water, facilitating the industrial production. The spinosin-Na monomer prepared herein has a purity greater than 98%, and thus it can be used as a reference standard. Moreover, the spinosin-Na is firstly demonstrated herein to be capable of significantly inhibiting the proliferation of various tumor cells and inducing the apoptosis of tumor cells, and thus it can be applied in the preparation of functional foods and/or medicines for preventing and/or treating tumors.

### BRIEF DESCRIPTION OF THE DRAWINGS

FIG. 1 is an HPLC chromatogram of Ziziphi Spinosae Semen polyphenol (ZSSP).

FIGS. 2A-B are liquid chromatograms of spinosin-Na and spinosin, respectively.

FIG. 3A is an MS¹ spectrogram of the spinosin-Na under a positive ion mode; FIG. 3B is an MS² spectrogram of the spinosin-Na under a positive ion mode; FIG. 3C is an MS¹ spectrogram of the spinosin-Na under a negative ion mode; and FIG. 3D is an MS² spectrogram of the spinosin-Na under a negative ion mode.

FIGS. 4A-B respectively show the effects of spinosin-Na and spinosin on the proliferation of tumor cells.

FIG. 5 shows an effect of the spinosin-Na on the proliferation of normal cells.

FIG. 6A is a flow cytometry map showing the effect of spinosin-Na on the apoptosis of the tumor cells; FIG. 6B shows the effect of spinosin-Na on the apoptosis of cervical cancer cells; and FIG. 6C shows the effect of spinosin-Na on the apoptosis of the colon tumor cells.

FIGS. 7A-B respectively show macroscopic appearances of livers from cMyc mice treated with the drug (n=6) or saline (n=6).

### DETAILED DESCRIPTION OF EMBODIMENTS

### EXAMPLE 1

### Preparation of spinosin-Na in Ziziphi Spinosae Semen

(1) Preparation of a crude polyphenol extract of Ziziphi Spinosae Semen

(1-1) Ziziphi Spinosae Semen was pulverized, and 20 g of the pulverized product was added to 200 mL of a pre-prepared acidic hydrolysis solution and hydrolyzed at 37° C. under shaking for 2 h, the acidic hydrolysis solution was a mixed solution of concentrated sulfuric acid and methanol in a volume ratio of 1:9. The hydrolyzed product was neutralized with 10 mol/L NaOH to terminate the hydrolyzation, and then centrifuged at 11,000 rpm for 10 min. The precipitate was discarded, and the supernatant was extracted with an equal volume of ethyl acetate and centrifuged at 11,000 rpm for 10 min to generate three layers. A lower aqueous phase was collected and repeatedly extracted 3 times. After the extraction, the aqueous phase was subjected to rotary evaporation at 60° C. to remove the ethyl acetate to produce the crude polyphenol extract of Ziziphi Spinosae Semen.

(1-2) A polyphenol content in the crude polyphenol extract of Ziziphi Spinosae Semen was measured by Folin-Ciocalteu assay, where gallic acid was used as a reference standard, and prepared into a series of standard solutions respectively with a concentration of 0, 0.02, 0.06, 0.1, 0.15, 0.2, 0.3, 0.4, 0.5 and 0.6 mg/mL. 100 μL of respective standard solutions and 400 μL of deionized water were added into a test tube, to which 100 μL of Folin-Ciocalteu reagent was added. The reaction mixtures were mixed uniformly and allowed to stand for 6 min for reaction. Then the reaction mixtures were respectively added with 1 mL of 7% Na₂CO₃ solution, mixed uniformly, allowed to stand for 90 min and measured at a wavelength of 760 nm. A standard curve was plotted by taking the absorbance as the abscissa and the gallic acid amount (mg) as the ordinate. The crude polyphenol extract of Ziziphi Spinosae Semen was treated in the same way as the standard solutions and then measured at 760 nm for the absorbance. By substituting the absorbance into the standard curve, the polyphenol content in the crude extract was determined to be about 508.25 mg.

(2) Separation and purification of the spinosin-Na in the crude polyphenol extract of Ziziphi Spinosae Semen

(2-1) The crude polyphenol extract of Ziziphi Spinosae Semen was loaded on an SP207 macroporous resin with a wet weight of about 30 g followed by static adsorption for 12-16 h. The microporous resin was eluted with ultrapure water and 30% acetone three times, respectively, and then eluted with 50% acetone. The eluate was collected, concentrated using rotary evaporation to remove the acetone and lyophilized to produce polyphenol powder of Ziziphi Spinosae Semen (ZSSP) which was stored at 4° C. for use.

(2-2) The ZSSP powder was dissolved in a 40% methanol solution to prepare a ZSSP methanol solution with a concentration of 5 mg/mL. The ZSSP methanol solution was drawn into a 1 mL syringe and filtered using a 0.22 um filter to an Agilent LC sample vial for use.

(2-3) A Phenomenex Hydro-RP C18 column (250 mm×10 mm, 4 μm) was correctly installed on an Agilent 1260 Infinity II HPLC, and then balanced respectively with 100% methanol and 40% methanol were for 30 min at a flow rate of 1 mL/min.

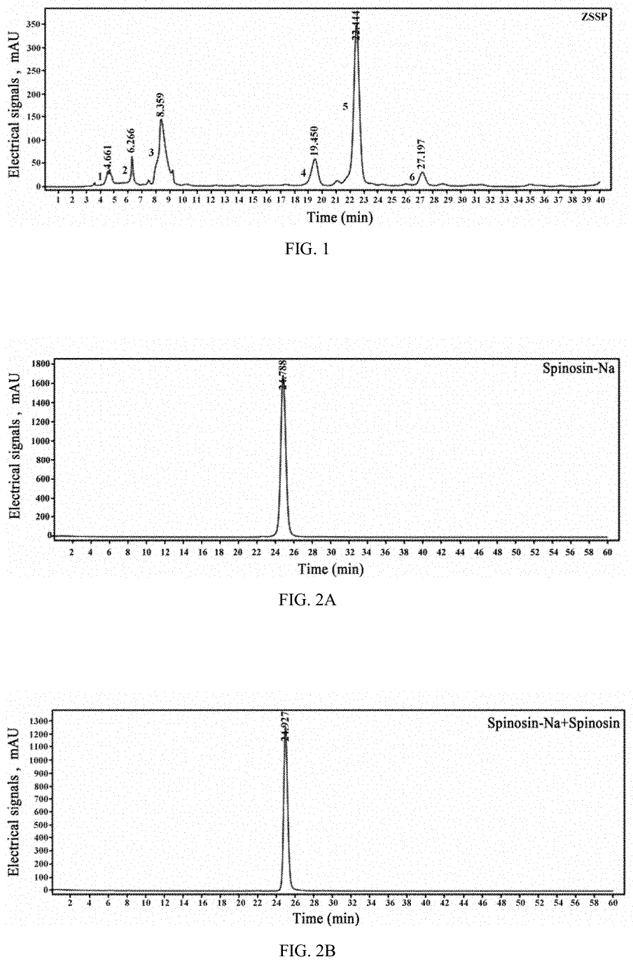

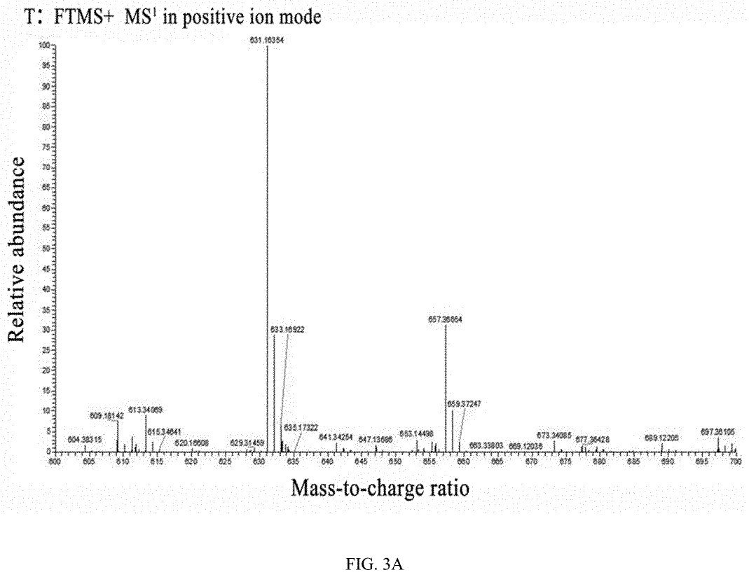

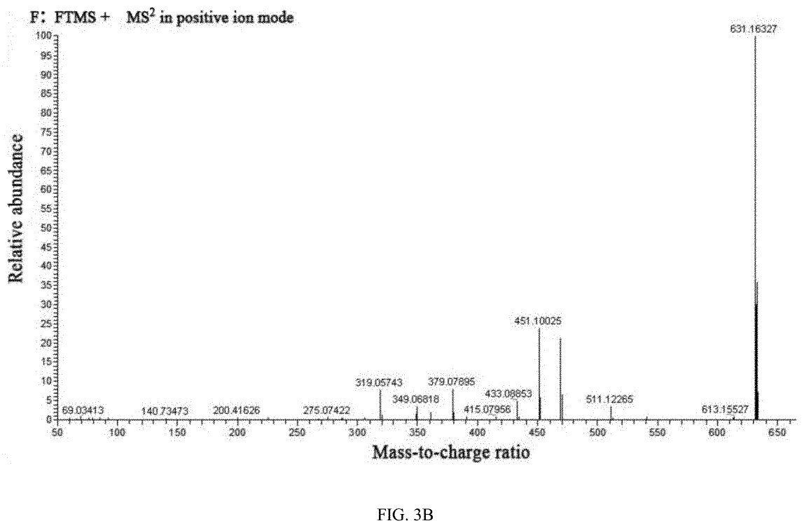

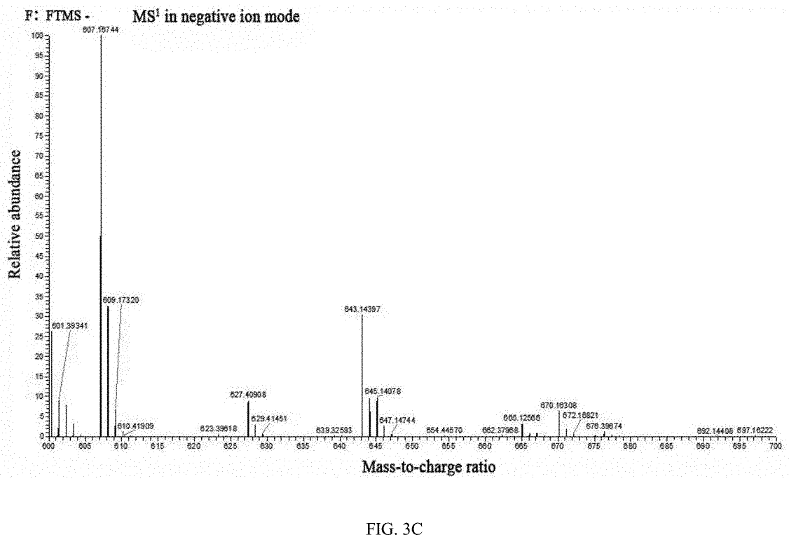

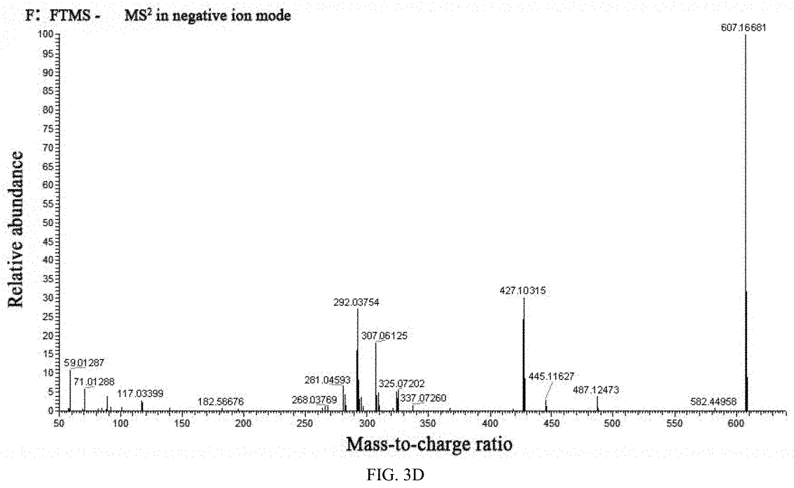

(2-4) 100 μL of ZSSP solution prepared in step (2-2) was loaded and analyzed at a wavelength of 330 nm under isocratic elution of 40% methanol solution. As shown in FIG. 1, there were six peaks on the obtained chromatogram. An eluting fraction with a retention time of 22.444 min (i.e., peak 5) was collected and identified to be spinosin-Na by MS using a spinosin standard (as shown in FIGS. 3A-3D), and the obtained spinosin-Na had a purity of 98.225% (as shown in FIGS. 2A and 2B).

Preparation of Spinosin-Na in a Large Amount

5 mL of ZSSP solution prepared in step (2-2) was loaded on a preparative HPLC (Shimadzu) equipped with a preparative Phenomenex Hydro-RP C18 column (250 mm×21.2 mm, 4 μm), where a mobile phase was 40% methanol solution; a measurement wavelength was 330 nm; and a flow rate was 10 mL/min. The eluting fraction of the peak 5 was collected, concentrated through rotary evaporation to remove the solvent and then lyophilized to produce spinosin-Na powder. The results showed that there was 43.2 mg of spinosin-Na monomer extracted from 20 g of Ziziphi Spinosae Semen.

### EXAMPLE 2

### Effect of Spinosin-Na of Ziziphi Spinosae Semen on Tumor Cell Proliferation

Colon cancer cell line HCT116, cervical cancer cell line Hela, breast cancer cell MCF-7, liver cancer cell HepG2 and normal colonic epithelial cell line FHC in the logarithmic phase were harvested and respectively made into a suspension. After counted, the suspensions were adjusted to 6000 cells/100 μL, and then 100 μL, of respective suspensions were seeded into a 96-well plate, and cultured at 37° C. and 5% CO₂ overnight. After that, the culture medium was discarded, and the wells were respectively added with culture mediums containing different concentrations of spinosin-Na, such that final concentrations of the spinosin-Na were respectively 50, 100, 150, 200 and 250 μg/mL. 5 parallel wells were set for each concentration treatment. The cells were continuously cultured in the incubator for 48 h. After that, the culture medium was discarded, and the cells were washed twice with PBS, added with 20 μL of 5.0 mg/mL MTT and continuously cultured for 4 h. The culture medium was discarded, and respective wells were added with 150 μL of DMSO to dissolve formazan crystals produced by living cells. The plate was shaken on a shaker for 10 min, and then transferred to a microplate reader for the measurement of absorbance at 570 nm.

The antitumor activity of different concentrations of spinosin (100, 200, 300, 400, 500 μg/mL) was measured by the same method described above.

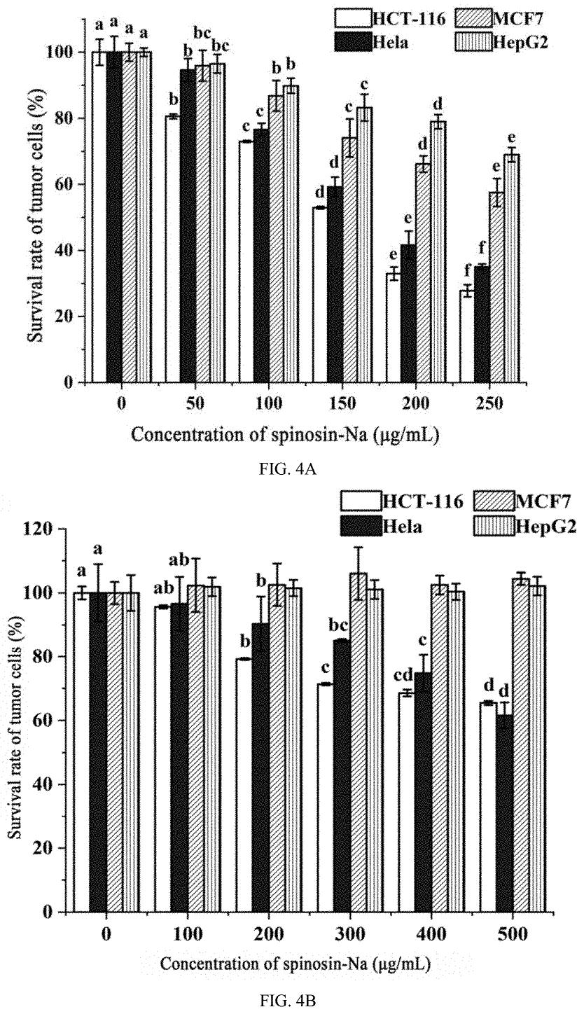

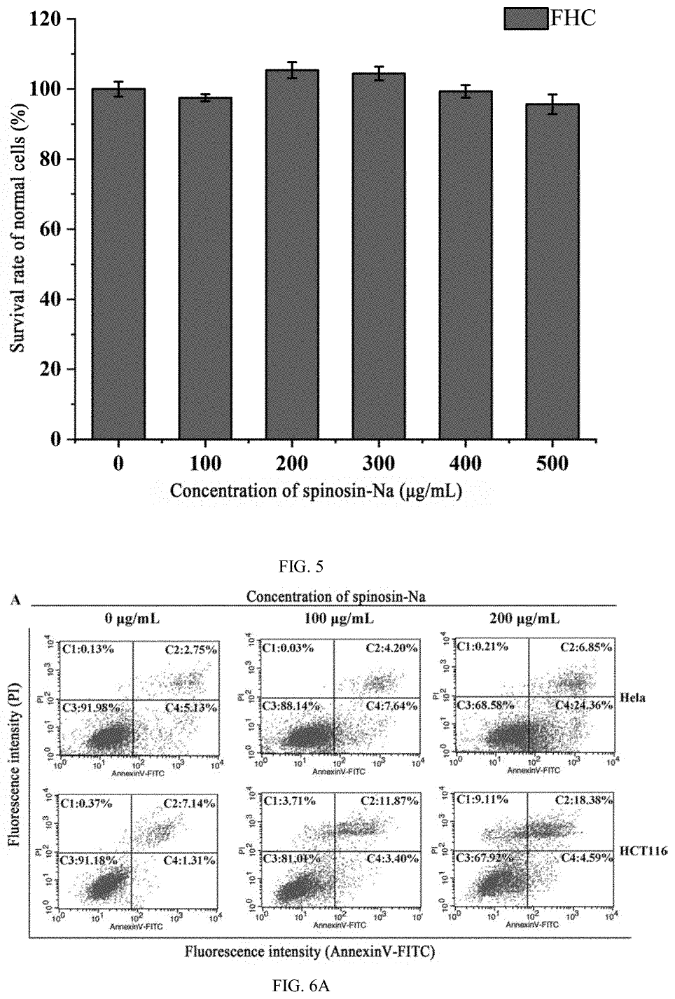

The in-vitro antitumor test results showed that spinosin-Na from Ziziphi Spinosae Semen could significantly inhibit the proliferation of tumor cells HCT116, Hela, MCF-7 and HepG2 (as shown in FIGS. 4A and 4B), and had no significant effect on the normal cell FHC (as shown in FIG. 5). Further, the inhibitory ability of spinosin-Na on tumor cells was significantly better than that of spinosin (as shown in FIGS. 4A and 4B and Table 1).

TABLE 1
IC₅₀ of spinosin and spinosin-Na for different tumor cell lines

| | | IC₅₀ (μg/mL) | |
|---|---|---|---|
| Cell line | | Spinosin-Na | Spinosin |
| Tumor cell line | HCT-116 | 147.20 ± 4.65 | 732.71 ± 18.97 |
| | Hela | 178.41 ± 6.83 | 687.60 ± 2132 |
| | MCF7 | 291.79 ± 10.49 | — |
| | HepG2 | 444.08 ± 15.21 | — |
| Normal cell line | FHC | — | — |

### EXAMPLE 3

### Effect of Spinosin-Na on Apoptosis of Tumor Cells

HCT116 and Hela cells in the logarithmic phase were harvested, digested with 0.25% trypsin, and seeded into a 6-well cell culture plate at a density of 1×10⁵ cells per petri dish. The cells were cultured at 37° C. and 5% CO₂ in an incubator overnight. After the cell adhesion was observed, the culture medium was replaced, and the dishes were respectively added with different concentrations of spinosin-Na. At the same time, a blank control was set, and each concentration treatment was performed in triplicate. The cells were continuously cultured in the incubator under the same conditions for 48 h. After that, the adherent cells were digested with 0.25% trypsin followed by centrifugation at 1,000 rpm for 5 min. The supernatant was discarded, and the cells were washed twice with PBS. The cells were suspended with 200 μL of a mixture of Annexin V and Binding buffer and reacted at 4° C. in the dark for 30 min. 300 μL of a mixed solution of PI and Binding buffer was added to each sample 5 min before the test. The double-stained cells were detected by flow cytometry to identify normal cells, early apoptotic cells, late apoptotic cells and necrotic cells, determining the apoptosis rate. Notes: the normal cells were labeled as AnnexinV−/PI−; the early apoptotic cells were labeled as AnnexinV+/PI−; the late apoptotic cells were labeled as AnnexinV+/PI+; and the necrotic cells were labeled as AnnexinV−/PI+.

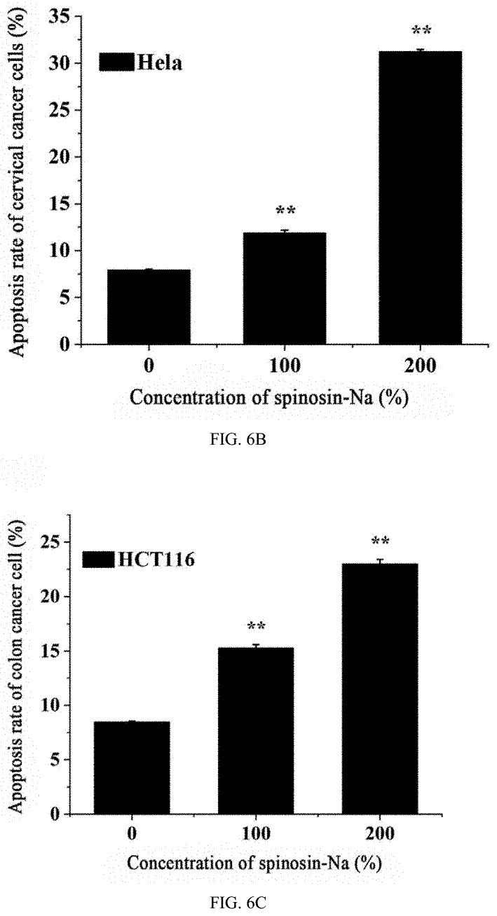

Flow cytometry results showed that the spinosin-Na could significantly induce the apoptosis of cervical cancer cells Hela and colon cancer cells HCT116 (as shown in FIGS. 6A-6C).

### EXAMPLE 4

### Inhibitory Effect of Spinosin-Na on Primary Liver Cancer in Mice

Mice were housed, fed, and monitored in accord with protocols approved by the Committee for Animal Research at the University of Minnesota (Minneapolis, Minn.). 10 μg of pT3-EF1α-cMyc and 0.4 μg of pCMV-SB plasmids were injected into the lateral tail vein of 6-week-old FVB/NJ mice in 5-7 s. At 4 weeks postinjection of cMyc, mice were randomly separated into two groups: group I mice (control group, n=6) were intraperitoneally injected with 100 μL of saline, group II (therapeutic group, n=6) were intraperitoneally injected with Spinosin-Na at a dose of 100 g/kg 1.5 mg once two days for 3 weeks.

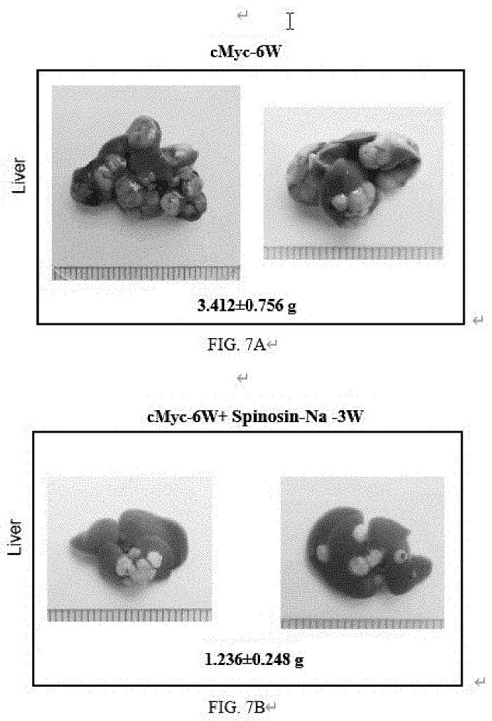

The data showed that spinosin-Na treatment significantly inhibited the growth of the liver tumor, and displayed a strong therapeutic effect on HCC in cMyc mice (as shown in FIG. 7).

What is claimed is:

1. A Spinosin-Na monomer of formula (I):

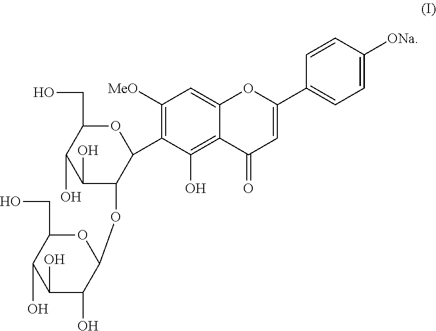

2. A method of preparing a spinosin-Na monomer from Ziziphi Spinosae Semen, comprising:
(1) pulverizing Ziziphi Spinosae Semen; hydrolyzing the pulverized Ziziphi Spinosae Semen with an acidic hydrolysis solution; neutralizing the hydrolysate with a NaOH solution to terminate the hydrolyzation; centrifuging the neutralized product and discarding the precipitate; extracting the supernatant with ethyl acetate followed by centrifugation to collect an aqueous phase as a crude polyphenol extract of Ziziphi Spinosae Semen; wherein a polyphenol content in the crude polyphenol extract of Ziziphi Spinosae Semen is measured by Folin-Ciocalteu assay;
(2) statically adsorbing the crude polyphenol extract of Ziziphi Spinosae Semen with a macroporous resin; eluting the macroporous resin with ultrapure water and 30% acetone three times, respectively; eluting the macroporous resin with 50% acetone to collect an eluate; concentrating the eluate by rotary evaporation at 60° C. until the eluate is decreased by 75% in volume; and drying the concentrated eluate under vacuum to produce polyphenol powder of Ziziphi Spinosae Semen (ZSSP);
(3) dissolving the ZSSP powder obtained in step (2) in 40% methanol to produce a ZSSP solution; finely separating the ZSSP solution using HPLC to collect a fraction with a retention time of 22.444 min; concentrating the fraction by rotary evaporation at 60° C., and lyophilizing the concentrated fraction to obtain the spinosin-Na monomer from Ziziphi Spinosae Semen.

3. The method of claim 2, wherein in step (2), the macroporous resin is an SP207 macroporous resin, and a static adsorption time is 12-16 h.

4. The method of claim 2, wherein in step (3), the HPLC separation is performed using a Phenomenex Hydro-RP C18 column (250 mm×10 mm, 4 μm) with a mobile phase of 40% methanol, a flow rate of 1 mL/min and a detection wavelength of 330 nm.

5. A composition comprising the Spinosin-Na monomer of formula (I) as defined in claim 1.

6. A method for treating tumors in a patient in need thereof, comprising administering to the patient an effective amount of the Spinosin-Na monomer of claim 1 as an active ingredient.

7. Use of the spinosin-Na monomer of claim 1 as a reference standard.
